(19) United States
(12) Patent Application Publication
(10) Pub. No.: US 2020/0390726 A1
(43) Pub. Date: Dec. 17, 2020

(54) Compositions for Improved Neuroprotective Effects and Methods of Making Same

(71) Applicant: Guardion Health Sciences, LLC, San Diego, CA (US)

(72) Inventor: Robert Ritch, San Diego, CA (US)

(21) Appl. No.: 16/899,384

(22) Filed: Jun. 11, 2020

Related U.S. Application Data
(60) Provisional application No. 62/859,885, filed on Jun. 11, 2019.

(51) Int. Cl. A61K 31/12; A61K 36/16; A61K 31/7068; A61K 31/122; A61K 31/198; A61K 31/385; A61K 36/87; A61K 36/82; A61K 31/4188; A61K 31/185; A61K 31/455; A61K 31/7084; A61K 31/353; A61K 31/7016; A61K 31/194; A61K 36/42; A61K 33/00; A61K 36/899; A61K 9/00; A61P 25/28; A61P 27/06; A23L 33/00; A23L 33/105; A23L 33/15; A23L 27/00; A23L 5/44; A23L 27/30; A23L 33/16

(52) U.S. Cl. CPC A61K 31/12; A23V 2002/00; A61K 31/7068; A61K 31/122; A61K 31/198; A61K 31/385; A61K 36/87; A61K 36/82; A61K 31/4188; A61K 31/185; A61K 31/455; A61K 31/7084; A61K 31/353; A61K 31/7016; A61K 31/194; A61K 36/42; A61K 33/00; A61K 36/899; A61K 9/0095; A61P 25/28; A61P 27/06; A23L 33/40; A23L 33/105; A23L 33/15; A23L 27/86; A23L 5/44; A23L 27/37; A23L 33/16; A61K 36/16

(57) ABSTRACT

A micro-nutrient composition for a human subject suffering from a glaucomatous disease, wherein the micro-nutrient composition comprises a formulation for reversing mitochondrial dysfunction in glaucomatous disease. Formulations for improving outcomes based on resolving conditions at the cellular response level in certain individuals suffering from particular diseases. More particularly, the invention relates to compositions affecting the eye, such as glaucoma.

### FIELD OF THE INVENTION

The invention relates to micronutrient formulations for improving outcomes based on resolving conditions at the cellular response level in certain individuals suffering from particular diseases. More particularly, the invention relates to compositions affecting the eye, such as glaucoma.

### BACKGROUND OF THE INVENTION

There are tens of millions of people that suffer from glaucoma worldwide, making it the second leading cause of blindness after cataracts. Glaucoma refers to a group of eye conditions that lead to optic nerve damage. The optic nerve carries visual information from the eye to the brain. Often time, damage to the optic nerve is the result of increased pressure in the eye, also known as intraocular pressure (IOP). The most common type of glaucoma is open-angle glaucoma which develops slowly over time with minimal to no pain to the individual. Peripheral vision loss is generally the first symptom, followed by central vision narrowing, resulting in total blindness if left untreated. Closed-angle glaucoma can present suddenly, which may involve severe eye pain, blurred vision, mid-dilated pupil, redness of the eye, and nausea. Vision loss from glaucoma, once it has occurred, is permanent.

Glaucoma is increasingly recognized as a neurodegenerative disorder, characterized by the accelerated loss of retinal ganglion cells (RGCs) and their axons. Open angle glaucoma prevalence and incidence increase exponentially with increasing age, yet the pathophysiology underlying increasing age as a risk factor for glaucoma is not well understood. Accumulating evidence points to age-related mitochondrial dysfunction playing a key role in the etiology of other neurodegenerative disorders including amyotrophic lateral sclerosis, Alzheimer and Parkinson disease. The 2 major functions of mitochondria are the generation of ATP through oxidative phosphorylation and the regulation of cell death by apoptosis. Research in the field suggests that age-associated mitochondrial dysfunction renders RGCs susceptible to glaucomatous injury by reducing the energy available for repair processes and predisposing RGCs to apoptosis (Kong et al., Mitochondrial dysfunction and glaucoma. J Glaucoma. February; 18(2): 93-100 (2009)).

Presently, there exist very few options to resolve the underlying conditions of glaucoma, especially if the disease has progressed to the point of near-blindness in the suffering individual. In fact, many of the goals of modern glaucoma management consist of avoiding further optic nerve damage in order to preserve visual field to the greatest extent possible while minimizing known side effects associated with elected course of treatment. Primary means of treatment range from the use of medications, usually in the form of eye drops, comprising prostaglandin analogs, beta-adrenergic receptor antagonists and mitotic agents to surgery options, including laser variants. However, each of the above treatment options comes with a unique set of local and systemic side effects, resulting in patient non-compliance with a given dosing regimen or lackluster results from post-operative assessments.

Alternatives to the aforementioned treatment options include certain nutritional supplements and other ingredients shown to improve vascular flow in order to reduce IOP, which may slow down the progression of visual field loss. Some of the currently marketed products available to patients suffering from glaucoma are provided below.

Anthocyanins: a type of antioxidant found in a number of types of fruit, including blueberry, blackcurrant, raspberries and apples, anthocyanins have been found to be a strong antioxidant. Such benefits of anthocyanins include the ability to lower IOP, reduction in time of visual field diminishment and a notable improvement of ocular blood flow when used at a daily dose of 50 mg (Wan et al., Survey of complementary and alternative medicine use in glaucoma patients. J Glaucoma. 21(2):79-82 (2012); Rhee et al., Prevalence of the use of complementary and alternative medicine for glaucoma. Ophthalmology. 109(3):438-443 (2002)).

Palmitoylethanolamide (PEA): PEA is an endogenous fatty acid amide involved in a number of biological functions related to inflammation and pain. Current work with PEA has shown a lowering of IOP, as well as an improvement in the visual field of patients, at a total dosing of 600 mg per day (Costagliola et al., Effect of palmitoylethanolamide on visual field damage progression in normal tension glaucoma patients: results of an open-label six-month follow-up. J Med Food. 17(9):949-954 (2014)).

Forskolin: Produced by the Indian Coleus plant, this labdane diterpene is a natural plant product and has been shown, through years of research, to increase levels of cyclic AMP (cAMP) through activation of the enzyme adenylyl cyclase. It is this ability of activating cAMP-sensitive pathways that results in decreased aqueous humor inflow and, thus, lowers IOP in glaucoma patients. An open label study recently showed a solution comprising 1% forskolin in eye drop formulation was sufficient to evidence a significant reduction in IOP and can thus be a safe alternative to beta blockers in glaucoma patients having concomitant asthma (Majeed et al., Efficacy and safety of 1% forskolin eye drops in open angle glaucoma—An open label study. Saudi J Ophthalmol. 29(3): 197-200 (2015)).

Citicoline: This is the generic name given to the endogenous compound cytidine-5′-diphosphocholine, which is known for its ability to increase neurotransmitter levels in the central nervous system through its interactions with phospholipid synthesis. Citicoline has proven neuroprotective effects in diseases ranging from Alzheimer disease to stroke, as well as providing a benefit to those patients with progressive glaucomatous disease in spite of well-controlled IOP (Iulia et al., Citicoline—a neuroprotector with proven effects on glaucomatous disease. Rom J Ophthalmol. 61(3): 152-158 (2017)). As a result, further glaucoma studies have shown a slowing of visual field deterioration when dosed around 500 to 1,000 mg daily (Roberti et al., Cytidine-5′-diphosphochline (citicoline) in glaucoma: rationale of its use, current evidence and future perspectives. Int J Mol Sci. 16(12): 28401-17 (2015)).

Mirtogenol®: A known dietary supplement consisting of a combination of bilberry extracts (Mirtoselect®) and French maritime pine bark (Pycnogenol®), this proprietary flavonoid blend has been shown to lower IOP, improve ocular blood flow and even improve the visual field of patients (Steigerwalt et al., Mirtogenol® potentiates latanoprost in lowering intraocular pressure and improves ocular blood flow in asymptomatic subjects. Clin Opththalmol. 4: 471-476 (2010)).

While the natural products and other non-surgical treatment options described supra may provide some therapeutic benefit, there is still a need in the art to reverse the underlying mitochondrial dysfunction associated with glaucomatous disease.

### SUMMARY OF THE INVENTION

The present invention provides a micro-nutrient composition for a human subject suffering from a glaucomatous disease, wherein the micro-nutrient composition comprises a formulation for reversing mitochondrial dysfunction in glaucomatous disease.

### DETAILED DESCRIPTION OF THE INVENTION

Mitochondrial dysfunction and death are increasingly implicated in retinal ganglion cell death in glaucoma and neuronal death in other neurodegenerative disorders. Mitochondria become dysfunctional and die prior to neuronal cell death.

Formulations with IOP-independent neuroprotective effects could be additive in slowing progression rates by reversing mitochondrial dysfunction. Furthermore, stabilizing mitochondria could shift the treatment paradigm to an earlier stage of disease (prior to retinal ganglion cell death) which would greatly improve therapeutic outcomes.

The following examples are intended to illustrate the invention by way of example only, and are not intended to limit the scope of the invention.

### EXAMPLE 1

### Randomized, Double-Blind, Placebo Control Trial on the Efficacy of a Novel Neuroprotective Combination in Glaucoma

A proprietary formulation was developed, which combined a cocktail of mitochondrial protectants with specific antioxidants to arrive at the final micro-nutrient composition for use in the treatment of glaucoma patients. The proprietary formulation comprised curcumin, Ginkgo biloba extract, citicoline, coenzyme Q10, N-acetyl-cysteine, alpha-lipoic acid, grape seed extract and green tea extract. A representative list of ingredients that make up the final micro-nutrient compositions as tested can be found below at Table 1.

TABLE 1
Micro-nutrient ingredient listing

| | Weight range |
|---|---|
| Component | (mgs) |
| Curcumin/curcuminoids | 400-600 |
| N-acetyl cysteine powder | 300-500 |
| Ginkgo biloba powder | 60-180 |
| R-lipoic acid (sodium salt) 80% | 50-150 |
| Citicoline | 50-150 |
| Grape seed extract powder (95%) | 50-150 |
| Green tea extract (98%) | 50-150 |
| Biotin (water soluble) 1% | 10-30 |
| L-taurine powder | 400-600 |
| Niacinamide powder (USP) | 200-300 |
| Fisetin | 25-75 |
| Quercetin (95%) | 50-150 |
| Luteolin | 50-150 |
| Nicotinamide adenine dinucleotide | 5-15 |
| Coenzyme Q10 | 50-150 |
| Dragon berry | 300-500 |
| Bitter blocker 10002185 | 200-400 |
| Citric acid powder (USP) | 400-800 |
| Sucralose | 100-200 |

To test the efficacy of this formulation, a trial was conducted on 14 patients (28 total eyes). The patients were randomized into two groups: Group I was given placebo and Group II was given the proprietary formulation.

Patients were tested with visual fields (VF), optical coherence tomography (OCT) and retinal metabolic analysis (RMA) at baseline, 1 month and 3 months post-randomization. RMA measures adjusted mitochondrial flavoprotein fluorescence (aFPF) and its average curve width (ACW), which are markers of mitochondrial oxidative stress. Hierarchical mixed effects linear models were tested for changes in VF, OCT and RMA indices over time.

As shown below at Table 2, the clinical characteristics of the treatment versus placebo groups are identified. None of the variables differed significantly between groups (P>5%).

TABLE 2
Demographic and clinical characteristics of the study sample

| Study Criteria | |
|---|---|
| Placebo | Formulation |

| Categorical | n | % | n | % |
|---|---|---|---|---|
| Gender (F) | 4 | 57 | 5 | 71 |
| Migraine | 1 | 14 | 1 | 14 |
| Sleep apnea | 4 | 57 | 3 | 42 |
| Raynaud's | 1 | 14 | 2 | 28 |
| Adverse events | 1 | 14 | 1 | 14 |
| Continuous | Median | IQR | Median | IQR |
| IOP (mmHg) | 17.75 | 6.00 | 13.50 | 7.00 |
| CDR | .70 | .40 | .70 | .30 |
| OCTmacH (microns) | 75.50 | 22.00 | 69.75 | 25.25 |
| OCTdisc (microns) | 68.00 | 11.00 | 76.50 | 28.00 |
| MD (dB) | −3.07 | 4.29 | −1.41 | 3.25 |
| PSD (dB) | 4.42 | 5.67 | 3.44 | 3.23 |
| VFI (%) | 93.50 | 15.00 | 96.50 | 8.00 |
| Fovea (dB) | 34.00 | 3.50 | 33.50 | 2.50 |
| HbA1C | 5.70 | .30 | 5.50 | .40 |
| LogMAR1 | .02 | .12 | .02 | .04 |
| Ocular meds | 1.00 | 2.00 | 3.00 | 3.00 |

During follow-up, there were no significant changes in VF (MD, PSD) or OCT (macular RGC) indices over time in either group (all P>5%). A significant decrease was shown in optic nerve aFPF and ACW from baseline to 1 month in the treatment group (β=−39, P=0.003 and β=−25, P=0.01, respectively) but not in patients treated with placebo (P=0.47 and 0.23) (Tables 3 and 4).

TABLE 3
Results of mixed effects linear models testing the
relationship between RMA parameters and change between
baseline and follow-up visit in treatment group

| Parameter | β | 95% Confidence Interval | | P-value |
|---|---|---|---|---|
| Optic disc aFPF | −39.78 | −66.30 | −13.26 | .003 |
| Optic disc ACW | −25.07 | −44.20 | −5.93 | .010 |

TABLE 4
Results of mixed effects linear models testing the
relationship between RMA parameters and change between
baseline and follow-up visit in placebo group

| Parameter | β | 95% Confidence Interval | | P-value |
|---|---|---|---|---|
| Optic disc aFPF | −15.54 | −57.75 | 26.66 | .470 |
| Optic disc ACW | −11.90 | −31.57 | 7.75 | .235 |

The results surprisingly showed that this proprietary formulation provided neuroprotective benefits to the tested class of patients. The above data evidence a reversal of mitochondrial dysfunction and should serve as a viable therapy for glaucoma patients.

An alternative embodiment to the above described, representative list of ingredients that make up the final micro-nutrient compositions of the present invention may also be in the following form described at Table 5:

TABLE 5
Alternative embodiment of micro-nutrient ingredient listing

| | Weight range |
|---|---|
| Component | (mgs) |
| Curcumin/curcuminoids | 400-600 |
| N-acetyl cysteine powder | 300-500 |
| Ginkgo biloba powder | 60-180 |
| R-lipoic acid (sodium salt) 80% | 50-150 |
| Citicoline | 50-150 |
| Grape seed extract powder (95%) | 50-150 |
| Green tea extract (98%) | 50-150 |
| Biotin (water soluble) 1% | 15-15 |
| L-taurine powder | 400-600 |
| Niacinamide powder (USP) | 200-300 |
| Nicotinamide adenine dinucleotide | 5-15 |
| Coenzyme Q10 | 50-150 |
| Dragon berry | 300-500 |
| Bitter blocker 10002185 | 200-400 |
| Citric acid powder (USP) | 400-800 |
| Astaxanthin | 1-10 |
| Flavonoid Complex | 200-300 |

In addition to the components mentioned supra, the alternative embodiment of the micro-nutrient composition may also, optionally, include one or more of the following: monk fruit, silica dioxide, soluble corn fiber and L-leucine. Preferably, the alternative embodiment of the micro-nutrient composition is formulated in a soluble powder form.

It will be appreciated that details of the foregoing embodiments, given for purposes of illustration, are not to be construed as limiting the scope of this invention. Although several embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

What is claimed is:

1. A micro-nutrient formulation for a human subject suffering from a disease, the formulation comprising:
a) curcumin;
b) Gingko biloba extract;
c) citicoline;
d) coenzyme Q10;
e) N-acetyl-cysteine;
f) alpha-lipoic acid;
g) grape see extract; and
h) green tea extract.

2. The formulation of claim 1 wherein the formulation is further comprised of biotin, L-taurine powder, niacinamide powder, nicotinamide adenine dinucleotide, dragon berry, bitter blocker 10002185, and citric acid powder.

3. The formulation of claim 2 wherein the formulation is further comprised of fisetin, quercetin, luteolin and sucralose.

4. The formulation of claim 2 wherein the formulation is further comprised of astaxanthin and flavonoid complex.

5. The formulation of claim 3 wherein the formulation is further optionally comprised of one or more of monk fruit, silica dioxide, soluble corn fiber and L-leucine.

6. The formulation of claim 4 wherein the formulation is further optionally comprised of one or more of monk fruit, silica dioxide, soluble corn fiber and L-leucine. The formulation of claim 3 wherein the formulation in a soluble powder form.

8. The formulation of claim 4 wherein the formulation in a soluble powder form.

9. The formulation of claim 3 wherein the disease is a neurodegenerative disease.

10. The formulation of claim 9 wherein the disease is glaucoma.

11. The formulation of claim 4 wherein the disease is a neurodegenerative disease.

12. The formulation of claim 11 wherein the disease is glaucoma.
